(19) United States
(12) Patent Application Publication
(10) Pub. No.: US 2007/0036673 A1
(43) Pub. Date: Feb. 15, 2007

(54) Fan-Driven Air Freshener

(76) Inventor: Raymond K. Selander, Hopewell Junction, NY (US)

Correspondence Address: INTERNATIONAL FLAVORS & FRAGRANCES INC., 521 WEST 57TH ST, NEW YORK, NY, 10019, US

(21) Appl. No.: 11/532,722

(22) Filed: Sep. 18, 2006

Related U.S. Application Data
(63) Continuation-in-part of application No. 10/682,051, filed on Oct. 9, 2003.

(51) Int. Cl. A61L 9/00

(52) U.S. Cl. 422/5; 422/124

(57) ABSTRACT

The present invention combines an air freshener that has a source of air freshening chemical with a fan that is controlled either by an optical device that senses light or a motion detector. When a light is turned on or motion is detected, the fan will be activated for a predetermined time period. In certain embodiments, the fan will stop turning after a predetermined time. Alternatively, the fan can continue to be powered until the light source is turned off (or all motion ceases), and only then either immediately shut down, or shut down after a predetermined time period. In certain preferred embodiments the source of air freshening chemical is disposed beneath the fan and allows fragrance to be delivered over time without the fan. The additional airflow provided by the fan causes more volatile fragrance chemicals to be removed from the source of air freshening chemical and admitted into the environment. The fan motor of the present invention is driven by a power source, such as batteries, AC line current or alternate sources such as solar cells. Preferably, a microprocessor controls the fan so that a “burst mode” is created by controlling the frequency and intensity of the pulses of air freshener that are emitted.

### STATUS OF RELATED APPLICATIONS

This application is a continuation-in-part of U.S. Ser. No. 10/682051 filed Oct. 9, 2003 which is a continuation-in-part of U.S. Ser. No. 11/069864 filed Mar. 1, 2005 the contents of all of which are all hereby incorporated by reference as if set forth in their entirety.

### FIELD OF THE INVENTION

The present invention relates to fragrance delivery systems, and more particularly to systems in which multiple fragrances are successively delivered into an environment in a pre-determined sequence.

### BACKGROUND OF THE INVENTION

Various devices are known that “freshen” air by adding a chemical to the air. In particular, off odors and malodors found in bathrooms are common. Various devices and chemicals that disinfect, i.e., kill odor-causing bacteria, or spray a perfume or fragrance to mask odors are known. Although many of these systems are passive and emit an air freshening compound into the air continuously, others use a fan to circulate the air freshening compound more rapidly and in higher concentration.

Currently available air fresheners with fans have various limitations. One limitation is that they do not deliver air freshening compounds effectively, primarily because the compound is delivered in intermittent bursts of varying intensity, or pulses, while the fan is operating. Additionally, currently available designs simply turn the fan on and off manually. If the fan is activated for a period of time beyond that needed the life of the fan and motor assembly is shortened unnecessarily, as is the battery life in battery-driven models. Moreover, air freshening chemicals volatilized by the fan are used up more quickly if the fan is either constantly running or running for a period of time longer than necessary.

U.S. Pat. No. 4,695,435—Spector discloses an air freshener device with a motor driven fan that is activated by a light being turned on, and is deactivated when the light is turned off.

U.S. Pat. No. 4,707,338—Spector discloses an air freshener device with a motor driven fan that is activated by a light being turned on, and is deactivated after a set period of time.

Neither of these prior art devices address the problems outlined above. Therefore, there remains a long-felt yet unmet need for providing enhanced levels of volatile air freshening or aroma chemicals in an effective and efficient manner. It would therefore be desirable to provide materials and methods that enhance the efficiency of fan driven air freshening systems. It would further be desirable to provide such improvements in a manner that permitted their application across a wide variety of situations and that permitted their implementation in a cost-effective manner.

### SUMMARY OF THE INVENTION

Accordingly, it has now been found that these and other problems found in the prior art can be overcome by an air freshener apparatus that has a source of air freshening chemical, a photocell and a fan assembly disposed in a housing adjacent the source of air freshening chemical. The fan is controlled by the optical sensor such that the fan motor is activated for a predetermined time period upon the photocell sensing a predetermined level of light. In preferred embodiments, the source of air freshening chemical is a wick, and most preferably, the wick is disposed beneath the fan and allows fragrance to be delivered over time without the fan. In certain embodiments, the air freshener also has a control circuit, or shutoff circuit that deactivates the fan motor after a predetermined time, or alternatively shuts the motor off if the sensor senses a level of light below a predetermined level, either immediately or after a predetermined length of time. The fan motor is either driven by direct current or AC line current. In the latter, in certain preferred embodiments, the housing comprises a plug that connects the motor to the AC line current via a wall outlet and a receptacle wherein the wall outlet retains its utility and can be used to power another device simultaneously with the fan.

In one aspect of certain preferred embodiments of the present invention, a microprocessor is connected to the fan motor, and drives the fan at a predetermined frequency for a predetermined duration. Most preferably, the microprocessor is connected to a micropump and to an electron spray device.

In alternate embodiments, the air freshener apparatus uses a motion sensor to control the fan. In these embodiments, the fan motor is activated for a predetermined time period upon the motion sensor being activated, and the device also has a shutoff circuit. In a manner similar to the optical sensor embodiments, the shutoff circuit either deactivates the fan motor after a predetermined time, which is either pre-set or determined by the absence of motion.

Delivery of two or more fragrances from the muti-fragrance cartridge to release a pleasant accord of fragrance at a given time period.

### BRIEF DESCRIPTION OF THE DRAWINGS

FIG. 1 is a side elevation view of a first embodiment of a fan driven air freshener made in accordance with the present invention;

FIG. 2 is a perspective view of a second embodiment of a fan driven air freshener made in accordance with the present invention.

FIG. 3 is a block diagram of the operation of a fragrance dispersal system utilized in conjunction with preferred embodiments of the present invention;

FIG. 4 is partially schematic illustration of the components used in a microfluidic spray device;

FIG. 5 is an elevation view of a micropump and capillary needle used in the device illustrated in FIG. 4; and

FIGS. 6A-6B are partially schematic views of a fragrance-emitting clock made in accordance with the present invention.

### DETAILED DESCRIPTION OF THE PREFERRED EMBODIMENTS

The implementation of the present invention is in several preferred embodiments, discussed below, along with several illustrative examples. The embodiments of the invention described below are provided for the purpose of understanding the invention and are not meant to be limiting.

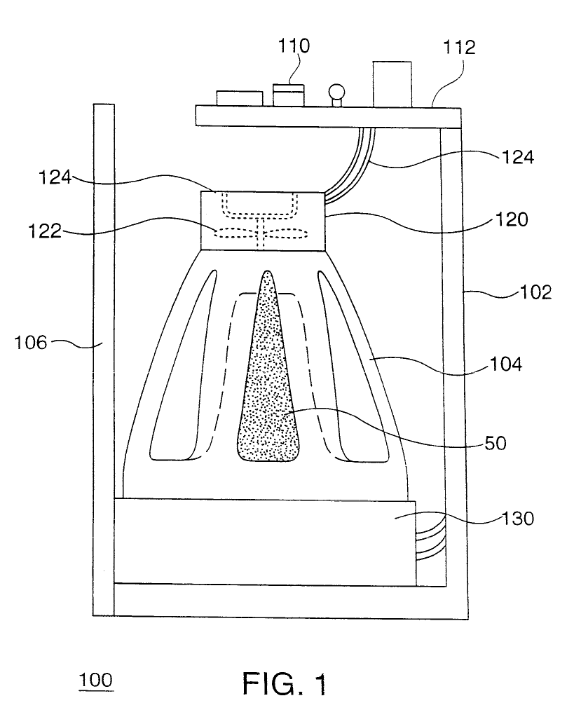

Referring now to FIG. 1, a side elevation view of a preferred embodiment of a fan driven air freshener 100 made in accordance with the present invention is illustrated. The apparatus is contained within a housing 102. In order to illustrate the invention those of skill in the art will understand that one side panel of the housing 102 has been removed. It will be further understood that the housing can be any of a number of designs and shapes, and is not limited to that which is illustrated. Generally, the housing 102 may be constructed from metal, plastic or any other suitable material that has sufficient structural strength to hold the components as shown while permitting sufficient airflow and that meets any safety or aesthetic criteria. Typically, but not necessarily, at least a portion of the housing 102 will comprise an air permeable panel 106 through which air may intermingle with air freshening chemicals. The chemicals are held in a reservoir 50, such as a wick, as is well known in the art. The chemicals may be any combination of odor masking or odor eliminating compounds that react with malodors or that have a more pleasant aroma than malodors. The composition and concentration of such chemicals for this use is well known. In a preferred embodiment, the reservoir 50 is contained within a shroud or reservoir housing 104. Preferably, the reservoir housing 104 allows the reservoir 50 to be handled without spilling or degrading the chemicals, and in certain embodiments may permit the chemicals to be replaced after they have dissipated by replacing the reservoir housing 104 and the reservoir 50 that contains fresh chemicals.

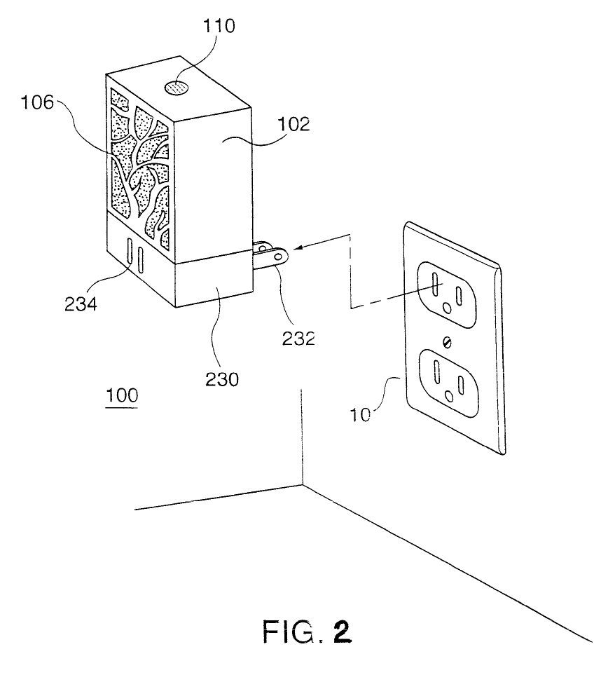

As illustrated in FIG. 1, a fan assembly 120 is preferably disposed above the reservoir 50 so as to force air through the apparatus. In certain preferred embodiments, placing the fan 120 over the reservoir 50 is preferred and is more effective than placing the fan 120 adjacent the reservoir 50. Moreover, such an embodiment can be constructed by modifying an existing air freshener assembly, which is less expensive than creating an entirely new assembly that positions the components elsewhere. The fan assembly 120 typically comprises a rotor 122 and a fan motor 124. Miniature fans suitable for any number of various embodiments of the present invention are readily available and easily adapted to the configuration shown in FIG. 1. The fan assembly 120 is driven by a power source 130. In the embodiment shown in IFG. 1 the power source 130 is preferably a direct current source, such as a battery. In addition to batteries, other conventional direct current power sources, such as solar cells, for one example, may be included in other embodiments. However, as explained in further detail below with reference to FIG. 2, the present invention also contemplates embodiments that use alternating current. The power source 130 is connected to a control circuit 112 by wires 124. As explained in further detail below, the control circuit 112 determines when the fan motor 124 is activated, and the duration of its activation.

In certain embodiments of the present invention, the control circuit 112 includes a sensor or photocell 110 that senses the level of light in the environment, and activates or deactivates a switch that supplies power to the fan motor 124. For example, the cell 110 can be chosen and put into a circuit so that the fan motor 124 is activated when a light is turned on in the room in which the apparatus is positioned. The control circuit 112 can also provide controls so that the fan 120 runs until the light is shut off, and then deactivates immediately. Alternatively, the fan 120 could run for a predetermined time (e.g., five minutes) or for a fixed time after the light source changes again, for example, when a light is turned off. The selection of a photocell and the components of the control circuit is conventional and well within the level of skill in the art. By running the fan motor 124 only when necessary, the component life is extended and the chemicals in the reservoir 50 are preserved.

Alternatively, in certain other preferred embodiments, the photocell 110 is replaced by a motion detector 110. In much the same manner as described in the preceding paragraph, the motion detector 110 determines when the fan assembly 120 should be activated, and in conjunction with the control circuit 112 determines how long a period of time the fan rotor 122 will turn. As mentioned above, the fan 120 is activated only when motion is sensed and shut off immediately in the absence of motion. Alternatively, the fan 120 can be activated when motion is sensed and then run for a fixed period. Finally, the fan can be activated and then run for a period of time measured after all motion has ceased. The selection of a motion detector and the components of the control circuit is conventional and well within the level of skill in the art.

Referring now to FIG. 2, and alternate embodiment of the present invention is illustrated. In this embodiment AC line current is used as a power source. As shown, the air freshener 100 contains an AC power supply 230 necessary to convert the line current and provide safety, if required, via a ground fault interrupter or similar circuit. The air freshener 100 has conventional plug prongs 232 that connect to a conventional receptacle 10. In the preferred embodiment illustrated in FIG. 2, a receptacle 234 is provided that is part of the power supply assembly 230 and plug prongs 232 that connects the assembly to the power source, so that the AC power outlet retains its utility and can be used to power another device simultaneously with the fan. Alternatively, in certain embodiments, particularly those intended for non-home use, the air freshener 100 is connected directly to an AC source via a junction box or similar wiring device and is thus permanently installed in terms of the electrical connection. The alternating current embodiment illustrated in FIG. 2 is otherwise identical to that described above with reference to FIG. 1.

In accordance with on aspect of the present invention, a “burst” mode of operation is provided. It has been found that by providing a microprocessor to control the operation of the fan described above, dramatic improvement in performance can be attained. In a most preferred embodiment, the flexibility of programming a microprocessor is utilized to its fullest advantage by incorporating a micro pump into the reservoir described above and driving the pump at a first frequency, and simultaneously driving an atomizing device such as an electro sprayer at a second frequency. The selection of ideal frequencies for any particular fragrance chemical combination is routine and does not require undue experimentation. However, in any embodiment, air freshener chemical will be introduced into the air even when the fan is deactivated. Experiments have shown that adding a burst mode to the above-described device can provide 2.8 times the evaporation (i.e., a 280% increase) an effect particularly well suited for bathrooms, where it is important to modify the air for short periods of time.

| System Type | Time (hr.) | Start Wt. (g) | End Wt. (g) | Rate (g/hr.) |
|---|---|---|---|---|
| Conventional | 16.2 | 217 | 215.6 | 0.00144 |
| Fan System: | 16.2 | 58.9 | 58.4 | 0.00052 |

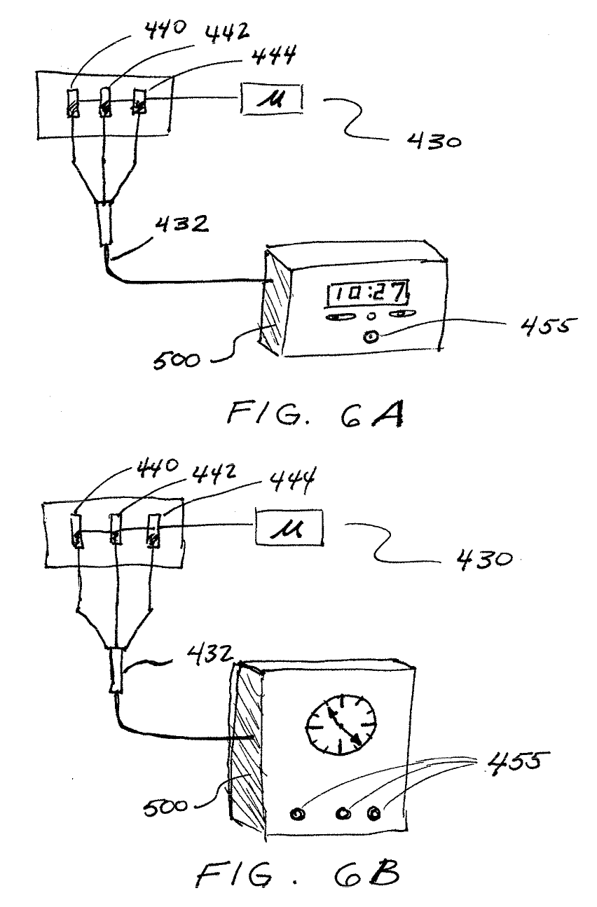

In other embodiments of the present invention, fragrances are delivered at specific time periods by either user selection or pre-selection. In preferred embodiments, the delivery is selected and arranged to tell a story, such as the scents one would encounter while for example walking in a park (floral scents, grass, dirt, wildlife, . . . ), or down a city street (bakery smells, florist shop, gas fumes, . . . ), etc. This selective delivery of fragrances provides an emotional experience relating to a pre-described story. In accordance with this aspect of the present invention, the fragrance selection is virtually unlimited. Multiple fragrances can be delivered at pre-selected time periods under the control of a programmed microprocessor or under the control of a programmed custom chip or ASIC. The multiple fragrances are preferably delivered at predefined consistent delivery rates over the life of the fragrance cartridge. The number of fragrances delivered may be as few as two, but is virtually limitless. Additionally, as described in further detail below with reference to FIGS. 6A-6B, by providing the ability to emit a number of fragrances, certain embodiments of the invention can be used to tell time (e.g., hourly) with a specific smell for each hour, i.e., different scents are emitted at hourly intervals and integrated with a digital clock or analog clock. For example, a user could wake up at 6:00 AM to a coffee fragrance, a noon break for lunch is signaled with a food scent, while the signal to return home might occur at 5:00 PM with a car interior scent.

In preferred embodiments, a micropump dispersal system will operate dependably in the context of the present invention without creating ozone or other undesirable atmospheric byproducts or “fallout,” yet delivering an even volumetric distribution of a fluid (i.e., a fine dispersal of fragrance) over the life of the device, or at a minimum until the volume of liquid in the reservoir is exhausted. In preferred embodiments of the present invention, a microfluidic spray device is incorporated and computer controlled to provide a fine spray of fragrance chemicals.

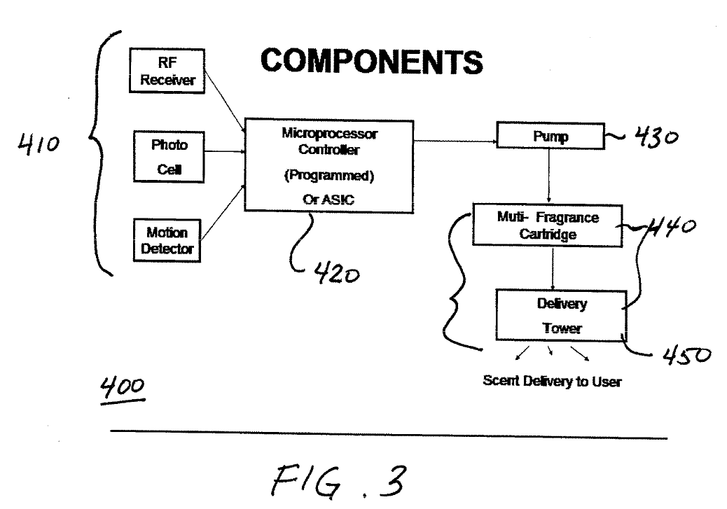

Referring to FIG. 3 a block diagram of the microfluidic dispersal system 400 integrated into certain preferred embodiments of the present invention is illustrated. An activation device 410 creates a signal that activates dispersal of one or more scents. As explained above, the activation device is typically but not necessarily an electronic signal and can be a timer or a clock, or a motion sensor or photocell. In any embodiment, a stimulus of some kind generates a signal, which in turn activates a microprocessor or an ASIC (Application Specific Integrated Circuits) 420, explained in further detail below, which operates the dispersal system. The integrated circuit in turn controls the operation of a micropump 430, which is preferably a piezoelectric device. The micropump 430 is connected to at least one source of fragrance chemical 440, but most preferably is connected to an array of fragrance chemicals, as explained below. The micropump 430 pressurizes the fragrance chemical 440 and the resulting atomized or volatilized fragrance is delivered to the environment or to the user, as explained with reference to other embodiments of the present invention. In preferred embodiments, the micropump 430 is a piezo electric device that provides constant volumetric flow over the life of the fluid supply in the fragrance chemical reservoir 440. Typically, electronic controls built into the microprocessor 420 provide precise voltage and frequency (in certain preferred embodiments the optimal frequency is 80 Hz) to the micropump 430 to deliver consistent volumetric delivery. Thus, as known in the art, the microprocessor 420 may provide for customized control of the operations of the micropump 430 and associated components, as well as providing options, such as on/off cycling, photocell operation, burst mode operation, motion activation and the like.

The microfluidic spray device is illustrated in FIG. 4. As shown, in preferred embodiments, multiple sources of fragrance chemicals 440,442,444 are provided and are connected to the pump 430 in a sequence determined by the microprocessor 420. In certain embodiments, the sequence will follow directly from the activation signal. For example, if there is a scene in a film that includes a vehicle crash, the smells of engine exhaust, burning rubber, gasoline and smoke might all sequentially follow from a single activation signal in the audiovisual source. In alternate embodiments, an activation signal might initiate a longer sequence of minutes or hours where a variety of scenes are dispersed in a sequence to tell a “story” via the scents themselves. In any embodiment, it is preferred that the micropump 430 connect vial a capillary tube 434 to a charged needle dispersal valve 452, which is described in greater detail below. The charged needle system provides an output of finely divided droplets of scent chemical that disperse evenly and volatilize the scent efficiently without resort to carrier gases or high pressure. Although the system described will volatilize a liquid into a fine dispersal, it is preferable in certain embodiments to add a fan 460 to accelerate and assist in the delivery of fine fragrance to fill a room.

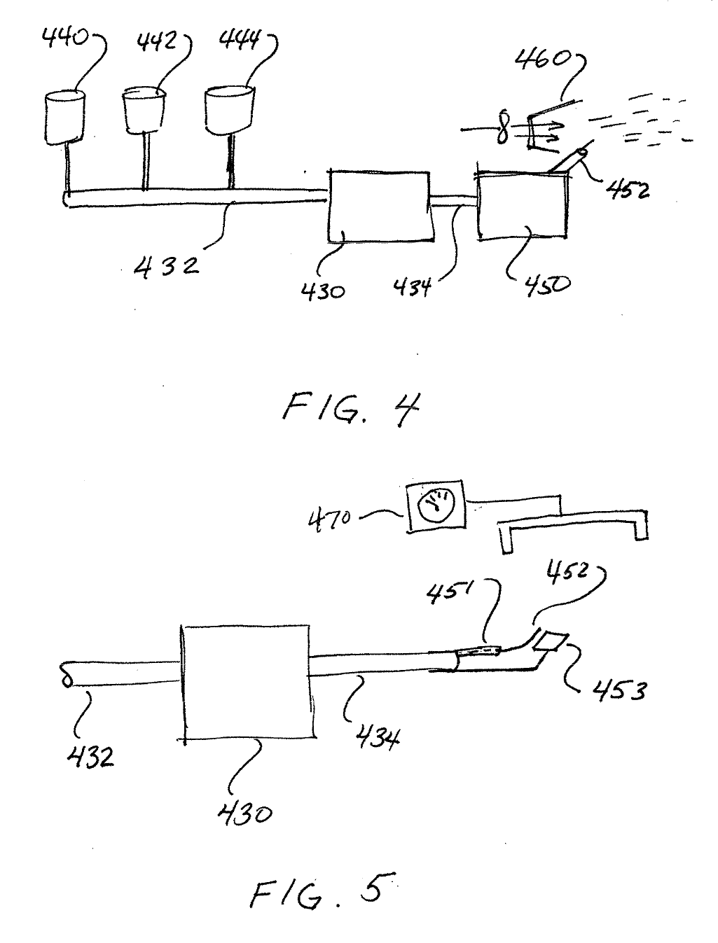

Further details of the microfluidic spray device are shown in FIG. 5. A tube 432 carries fragrance chemical into the pump 430 and, as mentioned above, a plastic capillary (tube) 434 of about 160 microns I.D. with a wall thickness of about 19 microns exits the pump 430. Inside this plastic capillary 434 is an internal (exposed to passing fluid) stainless steel metallic wire (conductor) 451 of about 19 microns O.D that terminates at its distal end as a dispersal needle 452. A positive direct current, created by 2500 volts DC or less, is applied to nebulize fine fragrance oils as well as other fluids (e.g. sanitizers such as tetraethylene glycol (TEG)) without the addition of carrier gases, which have the potential of creating ozone or other harmful byproducts. The system uses a metallic ground foil 453 to provide a target plane that attracts the positively charged nebulized fluid. In other embodiments, the ground foil is positively charged if the needle is negatively charged. This target plane 453 is preferably is maintained at a fixed distance between 0.25 inches and 0.5 inches from the exit port of the needle 452. The needle 452 is insulated from all other components such as the fragrance chemical reservoir 440 and micropump 430. Although the length of the needle 452 is not critical to the operation, in preferred embodiments it is approximately 0.25 inches or greater in length for ease of manufacturing. To fine tune the system, it is understood by those skilled in the art that the capillary length is part of the fluidic resistance. The needle 452 is readily made longer or shorter in correspondence with the diameter of the capillary 434 to reach the desired spray volume output. Preferably, the exit port of the capillary 434 and the end of the needle 452 have a blunt end (perpendicularly cut) that optimizes the nebulization process. The internal wire (conductor), inside the capillary, ends at the blunt end of the capillary. Materials that are compatible to fine fragrance oils are used in the implementation of our prototypes. They include Dupont Kalrez®, silicone and Polyphenylsulfone (PPSU). Materials that are avoided include Polycarbonate and Polystyrene, which are poor for handling fine fragrance oils.

Although calibration of the system described herein is readily accomplished with conventional available equipment is time consuming and not very accurate, it does not require undue experimentation. However, each fragrance oil used presents another set of parameters (viscosity, conductivity, surface tension, etc) all of which affect the performance of the system and require re-calibration. Therefore, in certain preferred embodiments, the system will include a microprocessor, photodiode array, and light source disposed in the vicinity of the needle 452. The calibration system 470 is shown graphically in FIG. 5. Using discrete electronic components to make a calibration system that is dependable and accurate and provides real time measurements to permit more efficient and precise calibration. In these embodiments, the microprocessor control preferably includes a circuit design that permits the dispersal system describe herein to perform reliably and consistently. Those embodiments using ASIC's (Application Specific Integrated Circuits) will permit miniaturization of the device allowing for battery powered embodiments. The newly available Chip MAX668 along with a Microprocessor (PIC18F1220) and new software algorithms programmed into the PIC provide an optional Sine Wave or Square wave to drive the Micropump (Piezo) through Pulse Wave Modulation (PWM).

An advantage of the electrostatic systems described above is that allergens and the like are destroyed when in the discharge of the device. When high voltage is applied to fragrance oils, which include for example TEG (triethylene glycol) an attraction to airborne particles results causing them to precipitate. Thus one mechanism of action is that if the fragrance contains TEG or similar compounds, bacteria would become attached to these molecules and be destroyed as the oils are nebulized by electrostatic action. In addition to bacteria, other organic (or possibly even inorganic) particulates such as tobacco smoke, dander and the like can be beneficially removed from the airflow by the electrostatic nebulization process described above.

Referring now to FIGS. 6A-6B there is shown a clock 500 that incorporates the fragrance emitting systems of the present invention. In FIG. 5A and digital clock with a single scent outlet 455 is illustrated. In FIG. 5B an analog clock with multiple scent outlets 455 is shown. However, embodiments such as digital clocks having multiple outlets and analog clocks with single outlets are contemplated and within the scope of the present invention. As seen in FIG. 5A, in those embodiments where a single scent outlet 455 is used, the multiple reservoirs 440,442,444 described above are connected via a manifold or the like to the single outlet. Alternatively, as seen in FIG. 5B it may be desirable to provide multiple scent outlets 455, each of which is directly connected to a corresponding scent reservoir 440,442,444. Most preferably, the clock 500 incorporates the dispersal system described above, which is compact and efficient.

Whether controlled by the time of day or otherwise, the user may select “scent programming” to provide a scent from a fragrance cartridge for each hour or for another period of time other than hourly, such as every 10 minutes.

In preferred embodiments, the micropump delivers multiple fragrances at pre-selected time periods under the control of a programmed microprocessor or a programmed ASIC. In certain embodiments a personal computer is used in conjunction with a radio frequency (RF) remote control to program the remote control.

Preferably, a microprocessor and support electronics with embedded custom software to provide user selection or pre-selection of fragrance per selected time period. (e.g., release coffee scent at wake up time 6:00 AM through 7:00 AM). The microprocessor is integrated with a pump (such as piezoelectric pumps or micropumps) and a low pressure compressor that is attached to a fragrance cartridge or individual fragrance reservoirs. In certain embodiments, a fan is used to dissipate the scent. In addition to a clock or other timer control as described above, a photo cell or motion sensor as described above may also be utilized. The integration of at least certain of these components allows the system to deliver a selected fragrance (one of eight) to provide an emotional experience through smell sense that tells a story.

Upon review of the foregoing, numerous adaptations, modifications, and alterations will occur to the reviewer. These will all be, however, within the spirit of the present invention. Accordingly, reference should be made to the appended claims in order to ascertain the true scope of the present invention.

What is claimed is:

1. Air freshener apparatus comprising:
a source of air freshening chemical, comprising an array of multiple scent chemicals of distinctive and different scents;
an activation device chosen from the group consisting of a photocell, a motion detector, a timer and a clock; and
a dispersal system connected to the activation device disposed in a housing adjacent the source of air freshening chemical that is controlled by the optical sensor,
wherein the dispersal system is activated by the activation device to successively disperse two or more scents in a pre-determined sequence.

2. The apparatus of claim 1, wherein the activation device successively disperses a series of scents upon initiation.

3. The apparatus of claim 1, wherein the activation device comprises a clock and successively disperses a series of scents at pre-determined time intervals related to the time of day.

4. The apparatus of claim 3, wherein the time intervals are hours.

5. The apparatus of claim 4, the activation device comprises a timer and successively disperses a series of scents at predetermined time intervals related to the elapsed time since the activation device was activated.

6. The apparatus of claim 1, further comprising a fragrance delivery system comprising a plurality of fragrance chemicals volatilized by a microfluidic dispersal system in communication with the controlled airflow, whereby at least one of said fragrance chemicals is volatilized and the volatilized fragrance is collected and sensed by a subject.

7. The apparatus of claim 6, wherein the fragrance delivery system further comprises a capillary tube and a dispersal needle.

8. The apparatus of claim 2, wherein the dispersal needle is electrically charged.

9. The apparatus of claim 1, wherein said plurality of fragrance chemicals is connected to the micropump via a common conduit.

10. The apparatus of claim 1, further comprising a fan.

11. The apparatus of claim 1, further comprising a calibration system disposed adjacent the fragrance delivery system.

12. The apparatus of claim 1, further comprising a microprocessor controller for controlling the sequence in which said fragrances are dispersed.

13. The apparatus of claim 12, wherein the microprocessor controller is triggered by an activation signal.

14. The apparatus of claim 12, wherein the microprocessor controller is an ASIC.

15. The apparatus of claim 8, wherein the chemical includes one or more constituents that attract particulate matter when exposed to the dispersal needle.

16. A method of delivering into an environment a substance chosen from one or more of a fragrance or antibacterial agent comprising the steps of:
spraying the substance at a predetermined time using a micropump,
passing a discharge stream through an electrostatic spray device to create a delivery stream, and
controlling the micropump and spray device with an electronic device that comprises a timer circuit.

17. The method of claim 14, wherein the electronic device is one of a microprocessor or ASIC.

18. The method of claim 14, wherein the micropump is a piezoelectric pump and further comprising the step of removing particulates from the environment.
